(19) United States
(12) Patent Application Publication
(10) Pub. No.: US 2025/0177282 A1
(43) Pub. Date: Jun. 5, 2025

(54) OZONATED OIL AND REMINERALIZATION COMPOSITIONS AND METHODS

(71) Applicant: Jennifer Jablow, Watermill, NY (US)

(72) Inventor: Jennifer Jablow, Watermill, NY (US)

(21) Appl. No.: 18/966,903

(22) Filed: Dec. 3, 2024

Related U.S. Application Data
(60) Provisional application No. 63/605,759, filed on Dec. 4, 2023.

(51) Int. Cl. A61K 8/92; A61Q 11/00

(52) U.S. Cl. CPC A61K 8/922; A61Q 11/00; A61K 2800/28; A61K 2800/592; A61K 2800/92

(57) ABSTRACT

The present disclosure relates to compositions used for healing dental surfaces, dental tissues and remineralization of teeth including compositions including a remineralization agent and an ozonated oil.

### FIELD

The present disclosure includes compositions and methods for healing teeth and other dental tissues and remineralizing teeth.

### BACKGROUND

Use of ozone in dentistry has been on the increase. The ozone molecule can deliver benefits in the oral cavity and dental tissues. Ozone can have healing properties, antibacterial properties, anti-inflammatory properties, biofilm penetration and prevention, and possible whitening from oxygenation action. The use of ozone in modern dentistry, especially for gum disease treatment, can have positive effects because it is a powerful disinfectant. Ozone has a unique antimicrobial effect and treats oral abscesses and ulcers; minor tooth decay, gingivitis, may prevent further gum disease; and reduces tooth sensitivity. It also can be used to speed up healing after a dental surgery, deep root planning and scaling and reduce sensitivity of exposed dentin. Ozone treatment is a holistic therapy that can increase the body's healing ability by stimulating circulation and oxygenation and enhancing an immune response. Ozone can clean wounds in bones and soft tissues working destructively against bacteria, fungi, and viruses and controls bleeding.

### SUMMARY

In one embodiment, a composition is provided. The composition includes an ozonated oil and a remineralization and anti-sensitivity agent.

In another embodiment, a method of remineralizing teeth is provided. The method includes applying a composition to one or more dental surfaces in the mouth of a patient, the composition including an ozonated oil and a remineralization and anti-sensitivity agent; swishing the composition in the mouth of the patient; and expelling the excess composition.

In another embodiment, a method of remineralizing teeth is provided. The method includes applying a composition to one or more dental surfaces including hard and soft tissues in the mouth of a patient using a brush or finger, the composition including an ozonated oil and a remineralization and anti-sensitivity agent.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Advantages and other aspects of the exemplary embodiments included herein will become apparent from the following detailed description and not as a definition of the limits of the disclosure, for which reference should be made to the appended claims. Additional aspects and advantages of the disclosure will be set forth in the description that follows, and in part will be obvious from the description, or may be learned by practice of the disclosure. Moreover, the aspects and advantages of the disclosure may be realized and obtained by means of the instrumentalities and combinations particularly pointed out in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “%”, “% by weight”, “weight %” and “wt %” are all intended to mean unless otherwise stated, percents by weight based upon a total weight of 100% end composition weight. Thus 10% by weight means that the component constitutes 10 wt. parts out of every 100 wt. parts of total composition.

The term “dental surface” means soft or hard tissue of the oral environment including gums or a natural tooth surface (e.g., dentin or enamel).

The term “dental tissue” means the hard and soft tissues of the mouth.

All of the embodiments included here are with the proviso that the sum of ingredients in the exemplary compositions does not exceed 100%.

Aspects of the present disclosure include embodiments including various compositions used for healing of dental surfaces, dental tissues and remineralization of teeth including compositions as well as associated methods that can include making the embodiments of the present disclosure and methods of using same a remineralization agent that may also be an anti-sensitivity agent and ozone in, for example, an oil, such as, for example, an ozonated oil that can be carried in a base in the form of an oil, gel or dentifrice (i.e., tooth paste).

The aspects of the present disclosure are directed to compositions as well as methods of making and using compositions of the present disclosure that dental benefits including healing of dental surfaces, dental tissues and remineralization of teeth using a remineralization agent and can include an ozonated oil. Compositions of the present disclosure include compositions and components thereof that are applied to dental surfaces.

Embodiments of the present disclosure can include a remineralization agent that may also be an anti-sensitivity agent. HAP (hydroxyapatite) is a dental agent that is capable of remineralizing enamel and is an anti-sensitivity agent. The HAP can be nano, micro or regular size in any shape or a blend of any combination of 2 or 3 types. The amount of HAP can be in the range of from about 0.1 wt % to about 20 wt % or about 1 wt % to about 10 wt %. In certain embodiments of the present disclosure, such a remineralizing agent can also include a phosphate compound, a calcium compound, a calcium phosphate compound, hydroxyapatite and a caseinate. The phosphate compound can include, for example, a monobasic phosphate compound, a dibasic phosphate compound, a tribasic phosphate compound, calcium glycerophosphate, and combinations thereof.

Hydroxyapatite (HAP) is a naturally occurring mineral that can help strengthen and restore teeth in several ways including, for example: remineralizing teeth: HAP can help rebuild tooth enamel, which protects teeth from cavities and decay; reversing small cavities: HA can help reverse small cavities; reducing tooth sensitivity: HAP can help reduce pain from tooth sensitivity; forming a protective layer: HAP can help form a protective layer on the enamel and cementum root surface; filling in enamel fissures: HAP can help fill in small enamel fissures that are vulnerable to tooth decay.

HAP may work in tandem with the ozonated oil since the ozone can work against the bacteria and remove biofilm which would allow the HAP to penetrate the enamel layer more favorably.

Calcium acetate is a dental agent that is also capable of remineralizing enamel (i.e., rebuilding tooth enamel). Calcium acetate is capable of delivering calcium directly to the tooth, which integrates into the enamel and repairs damage, i.e., microscopic damage.

Embodiments of the present disclosure can include ozone in that can be in an ozonated oil (also referred to as ozonide oil) such as olive oil, castor oil, coconut, hemp oil, sesame oil, sunflower oil, canola oil, avocado oil, grapeseed oil, black seed oil, argan oil, pumpkin seed oil, mct oil or any combination thereof, such as olive oil or coconut oil or any combination thereof to form an ozonated oil. The amount of ozonated oil in the embodiments of the present disclosure can be in the range of from about 0.5 wt % to about 95 wt %, 0.5 wt % to 30 wt % (i.e., 5% ozonated olive oil in, for example a solution w coconut oil).

Ozonated oils, for example, olive oil, castor oil, coconut, hemp oil, sesame oil, sunflower oil, canola oil, avocado oil, grapeseed oil, black seed oil, argan oil, pumpkin seed oil, mct oil, almond oil, castor oil, jojoba oil or any combination thereof can be produced using a variety of methods including, for example, ozonation, of different vegetable oils, such as castor oil (U.S. Pat. No. 1,210,949), olive oil (U.S. Pat. No. 2,356,062) and soybean oil (U.S. Pat. No. 3,504,038). Another example includes an ozone generator connected to an aerator that is placed in a glass jar containing oil. The aerator bubbles the ozone from the ozone generator through the oil to make an ozonated oil.

Ozonated oil is known to have an antibacterial effect on the dental surfaces of both gums and teeth. It is also known to help break up the biofilm and be effective in controlling the harmful bacteria. Studies have shown Olive oil that has been ozonized is an effective tool for combating gum disease and restoring gum health. It has also been shown in the prevention of enamel demineralization (caries) by application of ozonized olive oil: An in vivo study investigated the effect of ozonized olive oil gel on enamel the phenomenon of demineralization in people undergoing orthodontic treatment. The reversal of enamel demineralization after orthodontic treatment has been shown to be facilitated by the use of an ozonized olive oil gel.

This use of ozone in an oil (i.e., an ozonated oil) combined with a remineralization and anti-sensitivity agent can help patients when applied chairside in a dentist's office or at home to dental tissues. The goal is to promote or maintain healthy gums, promote healing, promote remineralization of enamel and reduce sensitivity. Another benefit can be brightening of the tooth color.

Embodiments of the present disclosure can also include additional ingredients such as, for example, at least one non-ozonated oil, at least one flavoring agent, at least one preservative and at least one sweetening agent.

Embodiments of the present disclosure may include one or more non-ozonated oil, such as, for example, olive oil, castor oil, coconut oil, hemp oil, sesame oil, canola oil, avocado oil, black seed oil, pumpkin seed oil, grapeseed oil, or sunflower, mct, almond, jojoba or castor. The amount of non-ozonated oil can be in the range of from about 5 wt % to about 97%.

Embodiments of the present disclosure may include one or more flavoring agents. Flavoring agents can include, for example, a mint oil (such as, for example, spearmint (e.g. spearmint native oil) or peppermint (e.g., peppermint oil)) and mixtures thereof. Flavoring agents can be in the range of from about 0.1 wt % to about 5 wt %, by weight of the composition.

Embodiments of the present disclosure may include one or more sweetening agents. Sweetening agents can include, for example, saccharin, dextrose, sucrose, sucralose, lactose, maltose, levulose, aspartame, Stevia, xylitol, sodium cyclamate, D-tryptophan, dihydrochalcones, acesulfame and mixtures thereof, in particular, Stevia, xylitol and mixtures thereof. Sweetening agents can be generally used at levels of from about 2 wt % to about 7 wt %, by weight of the composition, from 0.1 wt % to about 2 wt % or from about 2 wt % to about 3 wt %.

Embodiments of the present disclosure may include one or more antioxidants or preservatives, such as, for example, tocopherols (vitamin E). The amount of tocopherols can be in the range of from about 0.2 wt % to about 3 wt %.

Embodiments of the present disclosure can also include additional ingredients such as a chemical that reduces bacterial contamination, for example, grapefruit seed extract, Magnolia bark extract, citrus extracts or tea tree oil. A plant extract that can be used as a replacement for triclosan, a chemical that reduces bacterial contamination. However, some studies have shown that tea tree oil can cause antibiotic-resistant bacteria to develop. The amount of tea tree oil (or GSE, Magnolia bark extract, or citrus extracts) can be in the range of from about 0.05 wt % to about 5 wt %.

Embodiments of the present disclosure can also include additional ingredients such as an ingredient that can help with bad breath and tooth decay, for example, Magnolia bark extract, an ingredient that can help with bad breath and tooth decay. It's often found in mouthwashes, chewing gum, and toothpaste. The amount of Magnolia bark extract can be in the range of from about 0.05 wt % to about 5 wt %.

Embodiments of the present disclosure can also include additional ingredients such as an antimicrobial compound that may be effective against biofilms from caries-associated bacteria, such as, for example, citrus extract or grapefruit seed extract. The amount of citrus extract or grapefruit seed extract can be in the range of from about 0.05 wt % to about 5 wt %.

Embodiments of the present disclosure can also include additional ingredients such as an antioxidant and preservative, such as, for example, vitamin E, an antioxidant and preservative that can help extend the shelf life of products. The amount of vitamin E can be in the range of from about 0.05 wt % to about 5 wt %

A dentifrice embodiment of the present disclosure can include, for example, a toothpaste base such as a mixture of glycerin, calcium carbonate and or silica, cellulose or xanthan gum and or sorbitol, and a surfactant such as sodium methyl coco taurate or sodium lauroyl sarcosinate. Additional ingredients for dentifrice embodiments of the present disclosure can include, for example, xylitol, Stevia or monk fruit sweetener. Physical stain removal ingredients such as hydrated silica or calcium carbonate or a mix thereof.

An embodiment of the present disclosure includes an oil, i.e., the formula could be 5% ozonated olive oil, 90% coconut oil, 3% HAP, 1% xylitol 0.5% vitamin E, 0.5% peppermint oil.

An embodiment of the present disclosure includes 95% ozonated olive oil, 3% HAP, 1% xylitol, 0.5% peppermint oil, 0.5% vitamin E.

An embodiment of the present disclosure includes 5% organic ozonated coconut oil, 40% organic sesame oil, 46.5% organic sunflower oil and 1% organic peppermint oil, 2% hydroxyapatite and 0.5% vitamin E.

An embodiment of the present disclosure includes a gel formula including 2% hydroxyapatite, 1% CoQ10, 1% Stevia, 0.5% Mentha piperita (Peppermint Oil), 0.5% vitamin E (tocopherol), 5.0% ozonated olive oil, 88.5% vegetable glycerin, xanthan gum or cellulose or 88.5% of a mixture of at least two of vegetable glycerin, xanthan gum or cellulose, 1% xylitol, 0.5% calcium glycerophosphate and optionally, hyaluronic acid.

An embodiment of the present disclosure includes 5% ozonated olive oil w 2% HAP in a base of 20% coconut oil, 30% sunflower oil, 40% sesame oil, 2% xylitol, 0.5% peppermint oil, 0.5% vitamin E.

An embodiment of the present disclosure includes it could be 95% ozonated olive oil 2% HAP, 2% xylitol, 0.5% peppermint oil 0.5%, vitamin E.

An embodiment of the present disclosure includes 90% ozonated olive oil 5% coconut oil 2% HAP, 2% xylitol, 0.5% peppermint oil, 0.5% vitamin E.

Embodiments of the present disclosure may be in the form of a gel or a dentifrice. A gel can include, for example, a remineralization agent that may also be an anti-sensitivity agent and an ozonated oil as well as an appropriate gelling agent. Such gelling agents can include, for example, thickeners, gelling agents or combinations thereof. For example, embodiments of the present disclosure can be carried in a glycerin cellulose gel base. Thickeners or gelling agents can include, for example, inorganic, natural or synthetic thickeners or gelling agents. The amount of thickener and gelling agent can range from about 0.10% to about 45% by weight of the composition. Examples of thickeners and gelling agents can include inorganic thickening silicas such as: allantoin, xanthan gum, acacia gum, cellulose gum, an amorphous silica, for example Zeodent® 165 (Huber Corporation); Irish moss; iota-carrageenan; gum tragacanth; or polyvinylpyrrolidone. Dentifrice embodiments can also include such thickeners in the amount included above. Gel and dentifrice embodiments may also include surfactant and a polishing agent, such as a silica, a calcined alumina, sodium bicarbonate, calcium carbonate, dicalcium phosphate or calcium pyrophosphate and surfactants such as sodium lauroyl sarcosinate, Sodium Methyl Cocoyl Taurate, sodium cocoyl glutamate, cocamidopropyl betaine disodium cocoyl glutamate.

Additional ingredients for gel embodiments of the present disclosure can include, for example, Hap, Coq10, Stevia, allantoin, xylitol, ozonated oil, glycerin, xanthan gum, peppermint oil or any mint oil, vitamin E (tocopherol) and optionally, hyaluronic acid.

A dentifrice embodiment of the present disclosure can include, for example, a toothpaste base such as a mixture of glycerin, calcium carbonate and or silica, cellulose or xanthan gum and or sorbitol, and a surfactant such as sodium methyl coco taurate or sodium lauroyl sarcosinate. Additional ingredients for dentifrice embodiments of the present disclosure can include, for example, Hap, Coq10, allantoin, Stevia, xylitol, ozonated oil, glycerin, xanthan gum, peppermint oil, vitamin E (tocopherol).

Embodiments of the present disclosure also include methods of treatment using the embodiment compositions of the present disclosure. Such methods of treatment can include application of the embodiment compositions of the present disclosure with by a dentist in a dentist's office to one or more dental surfaces of a patient that are experiencing irritation (including, for example, irritation from bacteria or inflammation) and or sensitivity or by the patient themselves to one or more dental surfaces that are experiencing irritation (including, for example, irritation from bacteria or inflammation) or sensitivity from possibly erosion, loss of minerals or recession w root exposure and or dentin exposure. Embodiments of the present disclosure can include compositions that are administered by an applicator (e.g., a brush or finger) or dental tray or compositions in the form of mouth rinse. Application using a mouthrinse including embodiment of the present disclosure (e.g., oil based mouthrinses that has ozonated oil and HAP), in which the patient can, for example, swish, brush or rub the mouthrinse/gel/toothpaste composition in their mouth onto affected area to coat one or more dental surfaces of the mouth of the patient from about 2 to about 20 minutes followed by expelling or spitting out the excess of the composition. No rinsing or expelling is needed if the embodiment composition is applied by a brush or finger. If treatment using the embodiment composition is applied to the patient's dental surfaces by a wearing tray, the treatment can be for about 20 minutes after the wearing tray containing the composition is placed in the patient's mouth followed by removing the wearing tray from the patient's mouth and the patient spitting or expelling the excess composition from the patient's mouth where the patient should not eat or drink for about 30 minutes thereafter. If the embodiments of the present disclosure are used added to a typical toothpaste or on top thereof or the ingredients are in the toothpaste (ozonated oil and HAP), it can be used everyday 2 times a day with daily brushing. As an alternative, toothpastes or gels can including embodiment of the present disclosure can also be added to affected area and left on overnight.

Embodiments of the present disclosure (e.g., the gel, paste, or oil-based versions) can be applied directly to one or more dental surfaces and left on overnight. Embodiments of the present disclosure can also be added on top of typical toothpaste and brushed with once or twice a day.

Another embodiment of the disclosure relates to a kit for home use of a composition of an embodiment of the present disclosure.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental novel features of the disclosure as applied to the exemplary embodiments thereof, it will be understood that various omissions and substitutions and changes in the form and details of devices and methods illustrated, and in their operation, may be made by those skilled in the art without departing from the spirit or scope of the disclosure. Moreover, it is expressly intended that all combinations of those elements and/or method steps, which perform substantially the same function in substantially the same way to achieve the same results, are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. It is the intention, therefore, to be limited only as indicated by the scope of the claims appended hereto.

1. A composition comprising:
a. an ozonated oil; and
b. a remineralization and anti-sensitivity agent.

2. The composition according to claim 1, wherein the ozonated oil in the range of from about 0.5 wt % to about 95 wt %.

3. The composition according to claim 1, wherein the ozonated oil includes olive oil, castor oil, coconut oil, hemp oil, sesame oil, sunflower oil, canola oil, avocado oil, grapeseed oil, black seed oil, argan oil, pumpkin seed oil, mct oil, jojoba, oil, almond oil, or mixtures thereof.

4. The composition according to claim 1, wherein the remineralization and anti-sensitivity agent is HAP (hydroxyapatite).

5. The composition according to claim 4, wherein the amount HAP (hydroxyapatite) is in the range of from about 0.1 wt % to about 20 wt %.

6. The composition according to claim 1, wherein the composition is in the form of an oil.

7. The composition according to claim 1, wherein the composition is in the form of a gel.

8. The composition according to claim 1, wherein the composition is in the form of a dentifrice.

9. A method of remineralizing teeth, comprising:
a. applying a composition to one or more dental surfaces in the mouth of a patient, the composition including:
i. an ozonated oil and
ii. a remineralization and anti-sensitivity agent;
b. swishing the composition in the mouth of the patient; and
c. expelling the excess composition.

10. The method according to claim 9, wherein the ozonated oil in the range of from about 0.5 wt % to about 95 wt %.

11. The method according to claim 9, wherein the ozonated oil includes olive oil, castor oil, coconut oil, hemp oil, sesame oil, sunflower oil, avocado oil, canola oil, grapeseed oil, black seed oil, argan oil, pumpkin seed oil, mct oil, jojoba, oil, almond oil, castor oil, or mixtures thereof.

12. The method according to claim 9, wherein the remineralization and anti-sensitivity agent is HAP (hydroxyapatite).

13. The method according to claim 12, wherein the amount HAP (hydroxyapatite) is in the range of from about 0.1 wt % to about 20 wt %.

14. The method according to claim 9, wherein the composition further includes at least one non-ozonated oil, at least flavoring agent or at least one sweetening agent.

15. A method of remineralizing teeth, comprising:
applying a composition to one or more dental surfaces including hard and soft tissues in the mouth of a patient using a brush or finger, the composition including:
i. an ozonated oil; and
ii. a remineralization and anti-sensitivity agent.

16. The method according to claim 15, wherein the ozonated oil in the range of from about 0.5 wt % to about 95 wt %.

17. The method according to claim 15, wherein the ozonated oil includes olive oil, castor oil, coconut oil, hemp oil, sesame oil, sunflower oil, canola oil, avocado oil, grape seed oil, black seed oil, argan oil, pumpkin seed oil, mct oil, almond oil, jojoba oil, castor oil or mixtures thereof.

18. The method according to claim 15, wherein the remineralization and anti-sensitivity agent is HAP (hydroxyapatite).

19. The method according to claim 18, wherein the amount HAP (hydroxyapatite) is in the range of from about 0.1 wt % to about 20 wt %.

20. The method according to claim 15, wherein the composition further includes at least one non-ozonated oil, at least flavoring agent or at least one sweetening agent.
